(19) United States
(12) Patent Application Publication
(10) Pub. No.: US 2025/0339171 A1
(43) Pub. Date: Nov. 6, 2025

(54) ENDOVASCULAR SAFETY CAGE

(71) Applicant: KONINKLIJKE PHILIPS N.V., EINDHOVEN (NL)

(72) Inventors: Andrew Scherer, MAPLE GROVE, MN (US); Sooneon Bae, MAPLE GROVE, MN (US); Ryan Michael Sotak, COLORADO SPRINGS, CO (US)

(21) Appl. No.: 18/871,169

(22) PCT Filed: Jun. 1, 2023
(86) PCT No.: PCT/EP2023/064726

§ 371 Date: Dec. 3, 2024

Related U.S. Application Data
(60) Provisional application No. 63/350,441, filed on Jun. 9, 2022.

(51) Int. Cl. A61B 17/3207

(52) U.S. Cl. CPC A61B 17/320758

(57) ABSTRACT

An intravascular therapy device (10) includes an intravascular catheter (12): a helical cage (14) configured to be rotated by the intravascular catheter into a vascular occlusion disposed in a blood vessel of an associated patient: and a treatment device (16) configured to be moved to a position inside the helical cage while the helical cage is rotated into the vascular occlusion and further configured to be operated to treat the vascular occlusion while positioned inside the helical cage.

### FIELD

The following relates generally to the catheter arts, vascular therapy, lesion treatment arts, and related arts.

### BACKGROUND

Treatment of endovascular obstructions generally becomes more difficult with the increased age of the obstruction. As the obstruction becomes more chronic, it typically becomes larger, more rigid, and more strongly adhered to the affected vasculature. As these obstructions become more chronic, the effectiveness of standard atherectomy and thrombectomy tools decrease.

In the development of new tools to treat these chronic cases, increasing the aggressiveness (sharpness of contacting features, force of contact, speed of motion) of the tool provides improved efficacy. The aggressiveness of the tool needs to be limited however to prevent damaging the treated vessel. Maintaining the safety of the device often results in making it less aggressive to a point where it is no longer effective.

The following discloses certain improvements to overcome these problems and others.

### SUMMARY

In some embodiments disclosed herein, an intravascular therapy device includes an intravascular catheter; a helical cage configured to be rotated by the intravascular catheter into a vascular occlusion disposed in a blood vessel of an associated patient; and a treatment device configured to be moved to a position inside the helical cage while the helical cage is rotated into the vascular occlusion and further configured to be operated to treat the vascular occlusion while positioned inside the helical cage.

In some embodiments disclosed herein, a vascular therapy method includes inserting a helical cage into a blood vessel of an associated patient at a blood occlusion disposed in the blood vessel and screwing the helical cage into a vascular occlusion disposed within the blood vessel; after screwing the cage into the vascular occlusion, inserting a treatment device inside the helical cage; operating the treatment device while inserted inside the helical cage to apply therapy to treat the vascular occlusion; removing the treatment device from the helical cage; and removing the helical cage from the blood vessel.

In some embodiments disclosed herein, a vascular therapy system includes a treatment device; and a helical cage configured to be rotated into a vascular occlusion disposed in a blood vessel of an associated patient, the cage having a diameter smaller than a diameter of the blood vessel and having a size to accommodate the treatment device.

One advantage resides in providing a safety barrier between a blood vessel wall and a therapy treatment tool.

Another advantage resides in preventing trauma to a blood vessel wall.

Another advantage resides in enabling a broader range of treatment tools to be used to treat a clot in a blood vessel by using a safety barrier between a treatment tool and the blood vessel wall.

Another advantage resides in providing a removable safety barrier between a blood vessel wall and a therapy treatment tool.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates a vascular therapy device in accordance with the present disclosure.

FIG. 2 diagrammatically illustrates a side view of a helical safety cage of the device of FIG. 1.

FIG. 3 diagrammatically illustrates a perspective view of the helical safety cage of FIGS. 1 and 2 from a vantage close to the central axis of the helix.

FIG. 4 diagrammatically illustrates a method of performing a vascular therapy method using the device of FIG. 1.

### DETAILED DESCRIPTION

In an endovascular clot debulking procedure, clot material is removed using a debulking tool such as a mechanical cutter or a laser catheter performing laser ablation. In such procedures, a significant concern is the possibility that the mechanical cutting or laser ablation could cut into the blood vessel wall causing weakening or even rupture of the blood vessel wall.

The following discloses a helical endovascular safety cage that is designed to be rotated into the treatment site. The diameter of the safety cage is chosen to be slightly smaller than the diameter of the inner blood vessel lumen so that when threaded into the treatment site the clot material is mostly inside of the helical safety cage and the wall of the blood vessel lumen is outside of the safety cage. After the helical safety cage is thusly placed, the debulking tool is introduced and operated to debulk the clot material inside the safety cage, while the blood vessel wall is safely located outside of the safety cage. In the case of laser ablation, the working distance of the laser light is typically about a few tens of microns so that the helical safety cage provides adequate protection for the blood vessel wall.

The helical safety cage is suitably made of stainless steel, nitinol, or a shape memory polymer, for example. In the latter two cases, the nitinol or shape memory polymer is set in the designed helical shape, and can then be collapsed for storage in a lumen of the delivery catheter and expands to its set size when deployed. After deployment of the helical safety cage at the treatment site, the same or a different catheter can be used to deliver the debulking tool to the treatment site. After treatment is complete the helical safety cage is removed by rotating it in the opposite rotational direction from that used in deployment, and it is then drawn back into the lumen of the catheter.

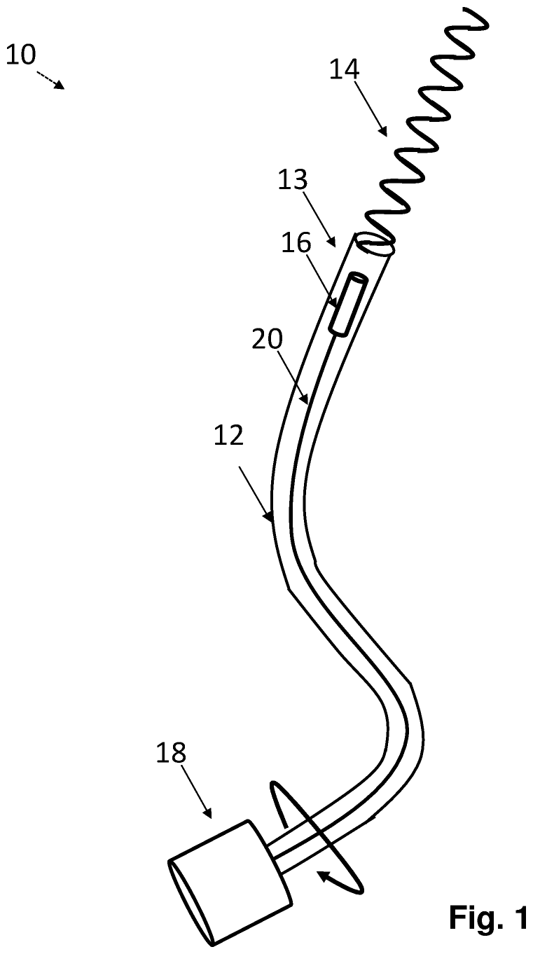

With reference to FIG. 1, an illustrative vascular therapy device 10 is diagrammatically shown. As shown in FIG. 1, the vascular therapy device 10 is insertable into a blood vessel for treating a lesion (or a clot, or an occlusion, and so forth) in the blood vessel. The vascular therapy device 10 includes, for example, an intravascular catheter 12, and a helical safety cage 14 disposed at a distal end 13 of the intravascular catheter 12 and configured to be rotated by the intravascular catheter 12 into a vascular occlusion disposed in the blood vessel. It is noted that FIG. 1 is not drawn to scale, and that the intravascular catheter 12 can have a length suitable to insert the catheter 12 into a blood vessel to deliver the distal end 13 through the vasculature along a (possibly tortuous) path to a treatment site, with the length of the catheter 12 being sufficient so that the rotary control 18 still remaining outside of the patient when the distal end 13 reaches the treatment site

A treatment device 16 (shown schematically in FIG. 1 as a cylinder) is configured to be moved to a position inside the helical safety cage 14 while the helical safety cage 14 is rotated into the vascular occlusion and further configured to be operated to treat the vascular occlusion while positioned inside the helical cage 14. The treatment device 16 is configured to be moved to the position inside the helical cage 14 while the helical cage 14 is rotated into the vascular occlusion and operated to treat the vascular occlusion using the same intravascular catheter 12 that is used to rotate the helical cage 14 into the vascular occlusion. In one embodiment, the treatment device 16 includes an occlusion debulking tool configured to fit within the helical cage 14 to debulk the vascular occlusion. In another embodiment, the treatment device 16 includes a laser ablation catheter configured to fit within the helical cage 14 to provide ablation therapy to the vascular occlusion.

A rotary control 18 disposed at a proximal end 15 of the intravascular catheter 12. The rotary control 18 is operatively connected to rotate the helical cage 14. The rotary control 18 can be manually rotated by a user, or can be motorized with a motor (not shown). In some embodiments, as shown in FIG. 1, a second intravascular catheter 20 disposed within the intravascular catheter 12 that coaxially surrounds the second intravascular catheter 20. The rotary control 18 is operatively connected to rotate the helical cage 14 by the second intravascular catheter 20. To do so, the second intravascular catheter 20 is configured to move the treatment device 16 to a position inside the helical cage 14 while the helical cage 14 is rotated into the vascular occlusion and to operate the treatment device 16 to treat the vascular occlusion.

It is noted that the illustrative catheter 12 can have other features not shown in FIG. 1, such as a guidewire lumen running the entire length of the catheter 12 for over the wire (OTW) delivery along a guidewire that is pre-inserted into the blood vessel along the path to the treatment site, or a shorter guidewire lumen with an exit port in a rapid exchange (RX) catheter design. By way of further illustration, other contemplated variants include addition of an ultrasound transducer at or near the distal end 13 for imaging the treatment site, addition of one or more radiopaque markers on the catheter 12 to enable visualization by a suitable interventional imaging modality, and/or so forth. In some embodiments, the proximal end may be connected with a vacuum pump (not shown) to implement vacuum aspiration via the sheath 22 or a lumen of the second intravascular catheter 20 to remove clot material cut away by the treatment device 16.

FIG. 1 depicts an embodiment in which the catheter 12 is an outer sheath that delivers the helical cage 14, and the treatment device 16 is mounted on the inner intravascular catheter 20 disposed within a lumen running through the outer sheath catheter 12. This design as substantial advantages. The sheath catheter 12 can be rotated by the handle or other rotary control 18 to rotate the helical cage 14 into place to protect the blood vessel inner wall. Thereafter both the helical cage 14 and the outer sheath catheter 12 can remain in place, with the helical cage 14 remaining attached to the distal end 13 of the outer sheath catheter 12, to provide a lined path through the vasculature within which the treatment device 16 can be operated via the inner intravascular catheter 20

However, in other embodiments (not shown) it is contemplated to use wholly separate and unrelated catheters for these operations. For example, a first catheter can be used to deliver the helical cage 14, and this first catheter is then withdrawn from the vasculature and a second catheter is inserted into the vasculature to deliver the treatment device 16, and then withdrawn and the first catheter reinserted to retrieve the helical safety cage 14. In such embodiments, the first catheter suitably includes a mechanically or electrically actuated clamp or the like operating as a release/pickup mechanism. Using this mechanism, the helical safety cage 14 is released after it is in place in the clot so that the first catheter can be removed while leaving the helical safety cage 14 in placed; and during the subsequent retrieval step the release/pickup mechanism recaptures the helical safety cage 14 to retrieve it.

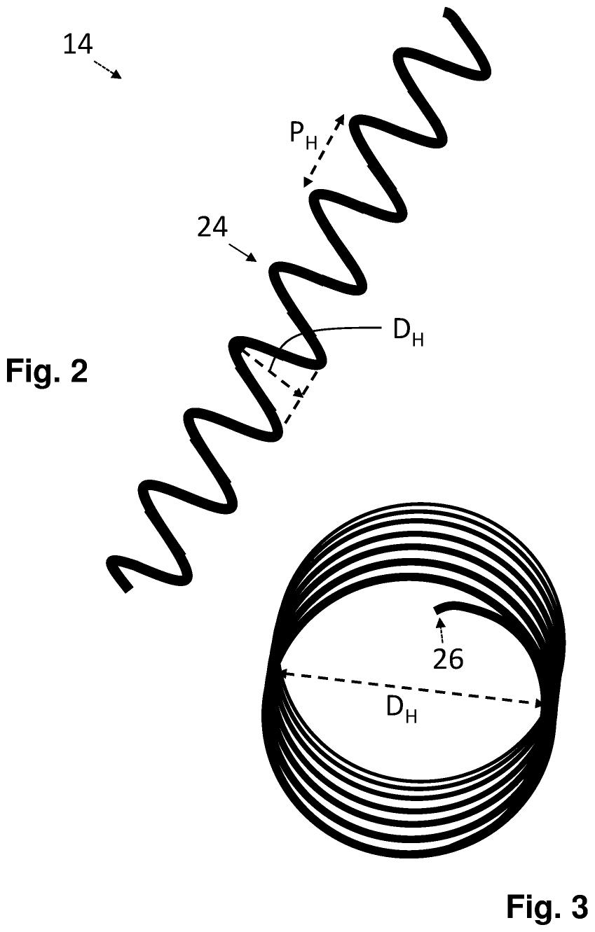

With reference to FIGS. 2 and 3, the helical cage 14 is shown in side view (FIG. 2) and in perspective view from a vantage close to the central axis of the helix (FIG. 3). The helical cage 14 has a diameter D_{H} indicated in FIGS. 2 and 3 that is preferably slightly smaller than the diameter of the blood vessel at the treatment site. The helical cage 14 also has a corresponding radius R_{H} not indicated in FIGS. 2 and 3, where R_{H}=0.5×D_{H}. The helical cage 14 is screwed into the clot material and hence is held in place by the clot material near the inner blood vessel wall. If the blood vessel has a diameter D_{BV} and corresponding vessel radius R_{BV}=0.5×D_{BV}, then the outermost portion of the clot corresponding to an annulus between the radii R_{H} and R_{BV} will be located outside of the helical cage 14 and hence will not be removed by the treatment tool 16 which operates within the helical cage 14. The choice of the diameter D_{H} of the helical cage 14 is suitably chosen based on the desired diameter of the core of clot material that is desired to be cut out (this is equal to D_{H}, neglecting the thickness of the stainless steel or other wire making up the helical cage 14) and the acceptable residual annulus of clot material between the radii R_{H} and R_{BV}. For therapeutic purposes, a typical design consideration is that the open lumen resulting from the therapy should have a diameter of about D_{H}, so this should be large enough to support the desired blood flow.

The helical cage 14 also has a helical pitch P_{H} labeled only in the side view of FIG. 2. It is noted that FIGS. 2 and 3 and diagrammatic, and that in particular the pitch P_{H} is shown in diagrammatic fashion. In general, the pitch P_{H}, along with the wire diameter of the stainless steel or other wire that is helically wound to form the helical cage 14, determines the size of the gap between adjacent turns of the helical coil 14. Neglecting the finite wire diameter this gap is thus equal to the pitch P_{H}. This gap is a space through via the treatment device 16 could in principle operatively pass in order to undesirably cut into the inner wall of the blood vessel. Hence, to avoid this undesirable cutting into the blood vessel wall, the pitch P_{H} should be selected to be small enough that (along with the actual wire diameter) the resulting gap is too small for the treatment device 16 to operatively penetrate. The appropriate pitch P_{H} can be chosen based on the nature of the treatment device 16. For example, in the case where the treatment device 16 is a laser ablation catheter, the physical size of the tip of the laser catheter will prevent the cutting laser aperture from passing through the gap between neighboring turns of the helical coil 14 and ablating the inner blood vessel wall if the pitch P_{H} is below some maximum permissible value. In this regard, it is again noted that the working distance of the laser light of a typical laser ablation laser aperture is typically about a few tens of microns. Similarly, if the treatment device 16 is a debulking tool employing a rotating or other type of cutter, then the size of the cutter and its mount to the distal tip of the inner catheter 20 will prevent the cutter from passing through the gap between neighboring turns of the helical coil 14 and cutting into the inner blood vessel wall if the pitch P_{H} is below some maximum permissible value.

The helical cage 14 can comprise, for example, stainless steel, Nitinol, or a shape memory polymer. The helical cage 14 comprises a plurality of turns 24 that form the helical cage 14. With particular reference to the near-end perspective view of FIG. 3, the helical cage 14 optionally has a distal end 26 that is turned inward toward an axis of the helical cage 14. This inwardly turned end 26 is the leading end as the helical cage 14 is screwed into the clot, and the inwardly turned end 26 reduces likelihood of the end being misdirected and embedding into the blood vessel wall.

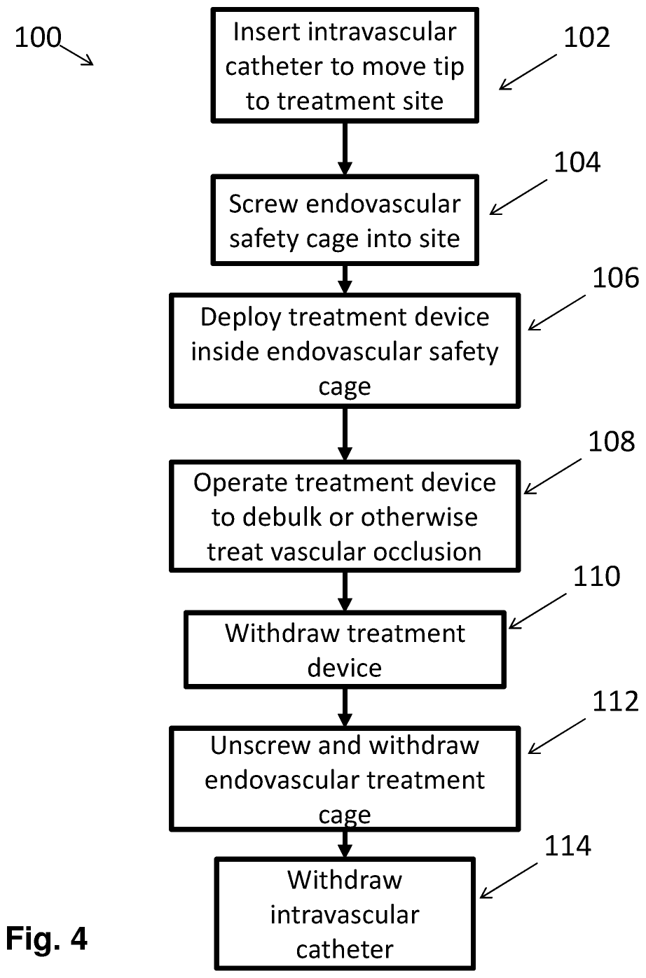

Referring to FIG. 4, an illustrative embodiment of an intravascular therapy method 100 using the intravascular therapy device 10 is diagrammatically shown as a flowchart. At an operation 102, the intravascular catheter 12 is inserted into a blood vessel to position the helical cage 14 proximate to a clot in the blood vessel. At an operation 104, the helical cage 14 is then screwed into the vascular occlusion. At an operation 106, the treatment device 16 is inserted inside of the helical cage 14. At an operation 108, the treatment device 16 is operated, while inserted inside the helical cage 14, to apply therapy (e.g., with an occlusion debulking tool, with a laser ablation catheter, and so forth). Once the therapy is complete, at an operation 110, the treatment device 16 is removed from the helical cage 14. At an operation 112, the helical cage 14 is removed from the blood vessel. At an operation 114, the intravascular catheter 12 is withdrawn from the blood vessel. As previously noted, using the device of FIG. 1 the operations 106, 108, and 110 can be performed with the outer sheath catheter 12 remaining in place and attached to the deployed helical safety cage 14, so that the additional steps performed by the human operator to use the helical safety cage 14 are only the additional steps 104 and 112.

The helical cage 14 should have sufficient density and thickness such that it prevents the treatment device 16 from contacting the vessel wall. For example, the wire diameter of the surgical stainless steel or other metal, ceramic, or polymer making up the helical safety cage 14 can be chosen to provide a sufficient safety barrier with sufficient stiffness to permit the helical safety cage 14 to be screwed into the clot. The helical cage 14 must also be durable so that contact with the treatment device 16 being activated inside of it will not cause damage. However, it is noted that the helical cage can be manufactured at low cost, and so in some embodiments is a consumable component that is only used for a single intravascular treatment. Hence, it need only be sufficiently durable for a single treatment session. Alternatively, if the helical cage 14 is made of surgical stainless steel or another durable material that is also autoclavable, then it could be sterilized between procedures and reused.

As shown in FIG. 3, one embodiment of the helical cage 14 includes a leading edge 26 that is angled inward towards the center of the vessel to reduce the likelihood of perforation when inserting the helical cage 14. The helical cage 14 can be collapsed down inside of the intravascular catheter 12, or employ shape memory action, so that it will fit through a smaller access size and be easy to navigate through the vessel. Once the tip of the intravascular catheter 12 reaches the treatment site, the helical cage 14 can be twisted out of the intravascular catheter 12 so that it expands to the desired size and threads itself between the obstruction and the vessel wall. Once the helical cage 14 is threaded into place, the treatment device 16 can be operated inside of the helical cage 14 with the risk of the treatment device 16 damaging the vessel wall being mitigated. Once the treatment is complete, the helical cage 14 can be rotated in the opposite direction to unthread/remove it from the vessel and return it to its collapsed state inside of the intravascular catheter 12 for removal from the body.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. An intravascular therapy device, comprising:
an intravascular catheter;
a helical cage configured to be rotated by the intravascular catheter into a vascular occlusion disposed in a blood vessel of an associated patient; and
a treatment device configured to be moved to a position inside the helical cage while the helical cage is rotated into the vascular occlusion and further configured to be operated to treat the vascular occlusion while positioned inside the helical cage.

2. The intravascular therapy device of claim 1, wherein the treatment device is configured to be moved to the position inside the helical cage while the helical cage is rotated into the vascular occlusion and operated to treat the vascular occlusion using the same intravascular catheter that is used to rotate the helical cage into the vascular occlusion.

3. The intravascular therapy device of claim 1, further comprising:
a second intravascular catheter different from the intravascular catheter;
wherein the second intravascular catheter is configured to move the treatment device to the position inside the helical cage while the helical cage is rotated into the vascular occlusion and to operate the treatment device to treat the vascular occlusion.

4. The intravascular therapy device of claim 1, wherein, when the treatment device is sized to fit within a diameter of the helical cage.

5. The intravascular therapy device of claim 1, wherein the helical cage comprises stainless steel.

6. The intravascular therapy device of claim 1, wherein the helical cage comprises Nitinol.

7. The intravascular therapy device of claim 1, wherein the helical cage comprises a shape memory polymer.

8. The intravascular therapy device of claim 1, wherein the treatment device includes:
an occlusion debulking tool configured to fit within the helical cage to debulk the vascular occlusion.

9. The intravascular therapy device of claim 1, wherein the treatment device includes:
a laser ablation catheter configured to fit within the helical cage to provide ablation therapy to the vascular occlusion.

10. The intravascular therapy device of claim 1, wherein the helical cage has a distal end that is turned inward toward an axis of the helical cage.

11. A vascular therapy method, comprising:
inserting a helical cage into a blood vessel of an associated patient at a blood occlusion disposed in the blood vessel and screwing the helical cage into a vascular occlusion disposed within the blood vessel;
after screwing the cage into the vascular occlusion, inserting a treatment device inside the helical cage;
operating the treatment device while inserted inside the helical cage to apply therapy to treat the vascular occlusion;
removing the treatment device from the helical cage; and
removing the helical cage from the blood vessel.

12. The vascular therapy method of claim 11, wherein applying therapy comprises:
applying the therapy with the treatment device comprising an occlusion debulking tool.

13. The vascular therapy method of claim 11, wherein applying therapy comprises:
applying the therapy with the treatment device comprising a laser ablation catheter.

14. A vascular therapy system, comprising:
a treatment device; and
a helical cage configured to be rotated into a vascular occlusion disposed in a blood vessel of an associated patient, the cage having a diameter smaller than a diameter of the blood vessel and having a size to accommodate the treatment device.

15. The intravascular therapy device of claim 14, wherein the helical cage has a distal end that is turned inward toward an axis of the helical cage.

16. The intravascular therapy device of claim 14 wherein the treatment device includes one of:
an occlusion debulking tool configured to fit within the helical cage to debulk the vascular occlusion; or
a laser ablation catheter configured to fit within the helical cage to provide ablation therapy to the vascular occlusion.

17. The intravascular therapy device of claim 14, wherein the helical cage comprises one of stainless steel, Nitinol, or a shape memory polymer.

18. The intravascular therapy device of claim 14, further including an intravascular catheter;
wherein the helical cage is configured to be rotated by the intravascular catheter into a vascular occlusion disposed in a blood vessel of an associated patient; and
the treatment device is configured to be moved to a position inside the helical cage while the helical cage is rotated into the vascular occlusion and further configured to be operated to treat the vascular occlusion while positioned inside the helical cage.

19. The intravascular therapy device of claim 18, wherein the treatment device is configured to be moved to the position inside the helical cage while the helical cage is rotated into the vascular occlusion and operated to treat the vascular occlusion using the same intravascular catheter that is used to rotate the helical cage into the vascular occlusion.

20. The intravascular therapy device of claim 18, further comprising:
a second intravascular catheter different from the intravascular catheter;
wherein the second intravascular catheter is configured to move the treatment device to the position inside the helical cage while the helical cage is rotated into the vascular occlusion and to operate the treatment device to treat the vascular occlusion.
